(19) United States
(12) Patent Application Publication
(10) Pub. No.: US 2024/0417409 A1
(43) Pub. Date: Dec. 19, 2024

(54) ORGANIC COMPOUNDS

(71) Applicant: GIVAUDAN SA, VERNIER (CH)

(72) Inventors: Eric EICHHORN, Kemptthal (CH); Thierry GRANIER, Kemptthal (CH)

(21) Appl. No.: 18/701,983

(22) PCT Filed: Oct. 20, 2022
(86) PCT No.: PCT/EP2022/079174

§ 371 Date: Apr. 17, 2024

(30) Foreign Application Priority Data
Oct. 21, 2021 (GB) 2115120.4

(51) Int. Cl. C07D 493/08; A61K 8/49; A61Q 15/00

(52) U.S. Cl. CPC C07D 493/08; A61K 8/498; A61Q 15/00

(57) ABSTRACT

The disclosure relates to sesquiterpene homologues, their use as fragrance and a method of their production.

### TECHNICAL FIELD

The present invention is concerned with fragrance ingredients and with fragrance preparations for imparting desired odor notes to consumer products. In particular it is concerned with a novel class of sesquiterpene homologues of formula (I) possessing woody ambery odor characteristics.

### BACKGROUND

In the flavour and fragrance industry there is a constant need for unique fragrance and fragrance compositions. For examples, there is a need for ingredients that are suitable for the fragrance industry possessing ambery woody odor notes. In modern perfumery ambery notes play a decisive role. They form the foundation of a lot of perfumes, and it is difficult to imagine a perfume without any woody or ambery notes.

It has now been found that sesquiterpene homologues as defined by formula (I) constitute ambery, woody odorants.

### SUMMARY

In accordance with a first aspect of the present invention there is provided a compound of formula (I)

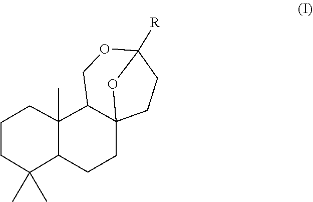

wherein R is selected from hydrogen, and C₁-C₄ alkyl (such as methyl, ethyl, n-propyl, and iso-propyl).

In accordance with a second aspect of the present invention there is provided a method for making a compound of formula (I) according to the first aspect of the invention, wherein the method comprises contacting a compound of formula (II) with a squalene-hopene cyclase (SHC) enzyme.

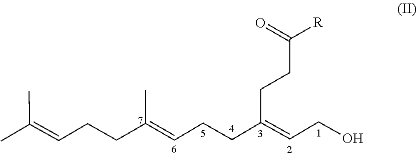

wherein R is selected from hydrogen, and C₁-C₄ alkyl (such as methyl, ethyl, n-propyl, and iso-propyl).

In certain embodiments, the method comprises contacting a compound of formula (II) wherein the double bond between C-6 and C-7 is in E-configuration and the double bond between C-2 and C-3 is in Z-configuration (E,Z-compound of formula (II)) with a squalene-hopene cyclase (SHC) enzyme (wild-type or variant enzyme).

In accordance with a third aspect of the present invention there is provided a composition comprising, consisting essentially of, or consisting of a compound of formula (I) and a compound of formula (III),

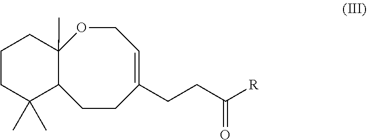

wherein R is selected from hydrogen, and C₁-C₄ alkyl (such as methyl, ethyl, n-propyl, and iso-propyl).

In accordance with a fourth aspect of the present invention there is provided a composition comprising, consisting essentially of, or consisting of a compound of formula (I), and a compound of formula (IV),

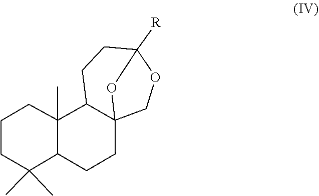

wherein R is selected from hydrogen, and C₁-C₄ alkyl (such as methyl, ethyl, n-propyl, and iso-propyl).

In certain embodiments of any aspect of the present invention R is methyl.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### SUMMARY OF THE SEQUENCES

SEQ ID NO: 1 is the amino acid sequence of wild-type Bacillus megaterium SHC (WT BmeSHC)

SEQ ID NO: 2 is the amino acid sequence of Alicyclobacillus acidocaldarius SHC enzyme variant #65 (AacSHC #65)

### DETAILED DESCRIPTION

The present invention is based, at least in part, on the surprising finding that compounds of formula (I) possess woody ambery odor characteristics. Furthermore, the invention is based on the surprising finding that squalene-hopene cyclase (SHC) enzymes can be used to make compounds of formula (I) from polyunsaturated alcohols of formula (II). It is particular surprising that a substrate wherein the alkenyl chain is substituted with an acylmethyl group as defined by formula (II) herein undergoes an enzymatic polycyclisation reaction terminated by internal ketalisation.

Thus there is provided in a first aspect a compound of formula (I)

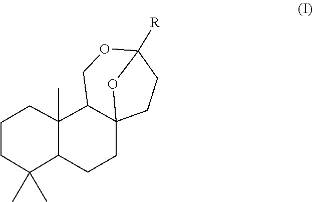

wherein R is selected from hydrogen, and C₁-C₄ alkyl (such as methyl, ethyl, n-propyl, and iso-propyl).

The compound of formula (I) contains a number of chiral carbon atoms and thus one or more stereoisomers of the compound of formula (I) may exist, including enantiomers and diastereomers.

In certain embodiments the compound of formula (I) may, for example, be a compound having the configuration of formula (Ia), wherein R is selected from hydrogen, and C₁-C₄ alkyl (such as methyl, ethyl, n-propyl, and iso-propyl):

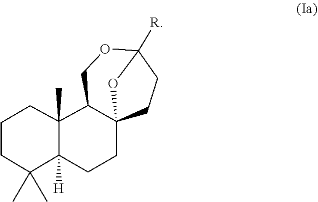

In certain embodiments R is methyl.

The compound of formula (I) may be made, for example, by an enzymatically mediated process.

The method provided herein enzymatically converts a compound of formula (II) to a compound of formula (I) (which encompasses a compound of formula (Ia)) using an SHC enzyme (bioconversion reaction).

Thus there is provided in a second aspect of the present invention a method of making a compound of formula (I) (which encompasses compounds of formula (Ia)) comprising the step of contacting a compound of formula (II) with a squalene-hopene cyclase (SHC) enzyme,

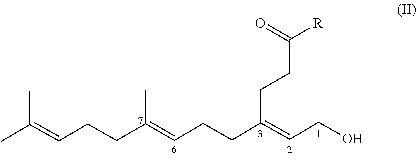

wherein R is selected from hydrogen, and C₁-C₄ alkyl (such as methyl, ethyl, n-propyl, and iso-propyl).

In certain embodiments the double bond between C-6 and C-7 is in E-configuration and the double bond between C-2 and C-3 is in Z-configuration (E,Z-compounds of formula (II)).

In certain embodiments both double bonds are in E-configuration (E,E-compounds of formula (II)).

In particular embodiments, R is methyl.

In certain embodiments, the method comprises contacting an E,Z-compound of formula (II) with a squalene-hopene cyclase (SHC) enzyme in the absence of any other stereoisomers of formula (II).

In certain embodiments, the method comprises contacting an E,Z-compound of formula (II) with a squalene-hopene cyclase (SHC) enzyme in the presence of an E,E-compound of formula (II), for example, the weight ratio of the E,Z-compound to E,E-compound of formula (II) may range from about 1:9 to about 9:1. For example, the weight ratio of the E,Z-compound of formula (II) to the E,E-compound of formula (II) may be from about 2:8 to about 8:2 or from about 4:6 to 6:4 (e.g. about 2:1).

In certain embodiments, the method comprises contacting a mixture comprising a compound of formula (II) and a constitutional isomer thereof with a squalene-hopene cyclase (SHC) enzyme.

Constitutional isomers of formula (II) are, for example compounds of formula (IIa)

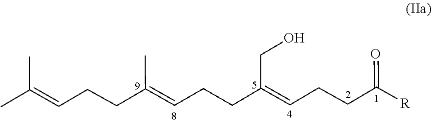

wherein R has the same meaning as for the compounds of formula (II) (i.e. R═H, or C₁-C₄ alkyl (such as methyl, ethyl, n-propyl, and iso-propyl)).

The compound of formula (IIa) exists in the form or four different stereoisomers, for example, as a compound of formula (IIa) having an E,E-configuration (compound of formula (IIa) wherein both double bonds are in E-configuration) or E,Z-configuration (compound of formula (IIa) wherein the double between C-8 and C-9 is in E-configuration and the double bond between C-4 and C-5 is in Z-configuration).

In certain embodiments, the method comprises contacting a mixture comprising, consisting essentially of, or consisting of E,E-compound of formula (II), E,Z-compound of formula (II), E,E-compound of formula (IIa), and E,Z-compound of formula (IIa). In one particular embodiment R is methyl.

The compound of formula (II) may be synthesized following the general procedure described in PCT/EP2022/066928 application.

The compound of formula (IIa) may be synthesized following the general procedure depicted by Fujiwara et al. (Tetrahedron Letters, 1995 Vol 36 (46), 8435-8438).

The bioconversion method described herein may, for example, make a compound of formula (III) as a by-product

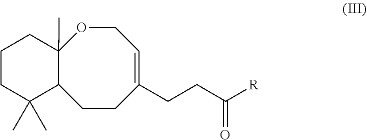

wherein R is selected from hydrogen, and C₁-C₄ alkyl (such as methyl, ethyl, n-propyl, and iso-propyl).

The compound of formula (III) may, for example, be a compound having the configuration of formula (IIIa)

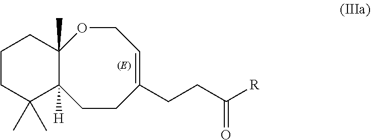

wherein R is selected from hydrogen, and C₁-C₄ alkyl (such as methyl, ethyl, n-propyl, and iso-propyl).

In particular embodiments, R is methyl.

The method described herein uses an SHC enzyme to enzymatically convert a compound of formula (II) to a compound of formula (I).

As used herein, the term “SHC enzyme” means both, a wild-type Squalene Hopene Cyclase enzyme that is naturally occurring in, for example thermophilic bacteria such as Alicyclobacillus acidocaldarius, and variant(s) of this SHC enzyme.

The term “variant” is to be understood as a polypeptide which differs in comparison to the polypeptide from which it is derived by one or more changes in the amino acid sequence. The polypeptide from which a variant is derived is also known as the parent or reference polypeptide. Typically a variant is produced artificially, preferably by gene-technological means. Typically, the polypeptide from which the variant is derived is a wild-type enzyme. However, the variants usable in the present disclosure may also be derived from homologs, orthologs, or paralogs of the parent polypeptide or from artificially constructed variants. The changes in the amino acid sequence may be amino acid exchanges (substitutions), insertions, deletions, N-terminal truncations, or C-terminal truncations, or any combination of these changes, which may occur at one or several sites.

In a particular embodiment, the SHC enzyme (e.g. from which the SHC enzyme may be derived—wild type or variant thereof) may be the Alicyclobacillus acidocaldarius (Aac) SHC enzyme, a Zymomonas mobilis (Zmo) SHC enzyme, a Bradyrhizobium japonicum (Bja) SHC enzyme, a Bacillus megaterium (Bme) SHC enzyme, or a Gluconobacter morbifer (Gmo) SHC enzyme. In a certain embodiment, the SHC enzyme (e.g. from which the SHC enzyme may be derived—wild type or variant thereof) may be the Alicyclobacillus acidocaldarius (Aac) SHC enzyme. In a certain embodiment, the SHC enzyme (e.g. from which the SHC enzyme may be derived—wild type or variant thereof) may be the Bacillus megaterium (Bme) SHC enzyme.

For ease of reference, the designation “AacSHC” may be used to refer to the Alicyclobacillus acidocaldarius (Aac) SHC enzyme, “ZmoSHC” may be used to refer to a Zymomonas mobilis (Zmo) SHC enzyme, “BmeSHC” may be used to refer to the Bacillus megaterium (Bme) SHC enzyme, “BjaSHC” may be used to refer to the Bradyrhizobium japonicum (Bja) SHC enzyme and “GmoSHC” may be used to refer to the Gluconobacter morbifer (Gmo) SHC enzyme.

AacSHC, ZmoSHC and BjaSHC enzyme sequences are disclosed in BASF WO 2010/139719, US 2012/01345477A1, Seitz et al (2012) J. Molecular Catalysis B: Enzymatic 84: 72-77) and Seitz (2012 PhD thesis, https://elib.uni-stuttgart.de/handle/11682/1400. Two different sequences are disclosed for ZmoSHC, referred to as ZmoSHC1 and ZmoSHC2. The GmoSHC enzyme sequence is disclosed in WO 2018/157021. Table 1 discloses sources and accession numbers of wild-type SHC enzymes.

TABLE 1
Sources and accession numbers of wild-type SHC enzymes.

| SHC Source Strain | | |
|---|---|---|
| (SHC name) | Reference | Accession No. |
| Alicyclobacillus | JP2009-060799 | NBRC15652 |
| acidocaldarius | Neumann et al Biol | |
| (WT AacSHC) | Chem (1986) 367; | |
| | 723-729 | |
| Zymomonas mobilis | WO2010139719 | ATCC31821 |
| (WT ZmoSHC) | US20120135477 | PF62207_2 |
| | | Genpept |
| | | Accession No |
| | | AAV90172 |
| Zymomonas mobilis | Reipen et al (1995) | EMBL/Genbank |
| (WT ZmoSHC) | Microbiology 141: | Accession No. |
| | 155-161 | X80766 |
| Bradryhizobium japonicum | WO2010139719 | PF62207_5 |
| (WT BjaSHC) | US2012/0135477 | |
| Bacillus megaterium | WO2017/150695 | WP_016763969.1 |
| (WT BmeSHC) | WO2019/045058 | |
| | WO2015/033746 | |
| Burkholderia ambifaria | WO2010139719 | |
| Bacillus anthracis | US2012/0135477 | |
| Frankia alni | | |
| Rhodopseudomonas palustris | | |
| Gluconobacter morbifer | WO 2018/157021 | |

The bioconversion methods of the present disclosure are carried out under conditions of time, temperature, pH and solubilizing agent (if used) to provide for conversion of the compound of formula (II) to the compound of formula (I).

The method for making the compound of formula (I) disclosed herein may be carried out at the optimum temperature range or optimum temperature and/or the optimum pH range or optimum pH and/or solubilisation agent (if used) optimum concentration range or optimum solubilisation agent concentration for the specific enzyme used.

The pH of the reaction mixture may be in the range of 4-8, preferably, 4.5 to 6.5, more preferably 4.5-6.5 for the SHC wild-type enzyme or SHC enzyme variant considered and can be maintained by running the reaction in an appropriate buffer, or adjusting pH during the time course of the reaction. An exemplary buffer for this purpose is a citric acid buffer, or a succinic acid buffer.

The temperature may be between from about 15° C. to about 60° C., for example from about 15° C. to about 50° C. or from about 15° C. to about 45° C. or from about 30° C. to about 60° C. or from about 35° C. to about 55° C. for the SHC enzyme. The temperature can be kept constant or can be altered during the bioconversion process.

It may be useful to include solubilizing agent(s) (e.g., surfactant, detergent, solubility enhancer, water miscible organic solvent and the like) in the bioconversion reaction. Examples of surfactants include but are not limited to Triton® X-100, Tween® 80, taurodeoxycholate, sodium taurodeoxycholate, sodium dodecyl sulfate (SDS), and/or sodium lauryl sulfate (SLS). In one particular embodiment SDS is used as solubilizing agent.

The compounds of formula (I) (which encompasses compounds of formula (Ia), in particular a compound of formula (Ia) wherein R is methyl) possess very interesting woody ambery odor notes. They may be used alone, or in combination with known odorant molecules selected from the extensive range of natural products, and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in fragrance compositions, for example, carrier materials, and other auxiliary agents commonly used in the art.

As used herein, “carrier material” means a material which is practically neutral from an odorant point of view, i.e. a material that does not significantly alter the organoleptic properties of odorants.

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same. It might also be an ingredient that provides additional benefits such as imparting color or texture. It might also be an ingredient that imparts light resistance or chemical stability to one or more ingredients contained in a fragrance composition. A detailed description of the nature and type of adjuvants commonly used in fragrance compositions containing same cannot be exhaustive, but it has to be mentioned that said ingredients are well known to a person skilled in the art.

As used herein, ‘fragrance composition’ means any composition comprising a compound of formula (I) (which encompasses compounds of formula (Ia)), or a mixture thereof and a base material, e.g. a diluent conventionally used in conjunction with odorants, such as diethyl phthalate (DEP), dipropylene glycol (DPG), isopropyl myristate (IPM), pentane-1,2-diol, triethyl citrate (TEC) and alcohol (e.g. ethanol). Optionally, the composition may comprise an anti-oxidant adjuvant. Said anti-oxidant may be selected from Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

The following list comprises examples of known odorant molecules, which may be combined with a compound of formula (I) (which encompasses compounds of formula (Ia)), or a mixture thereof:
essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil and/or ylang-ylang oil;
alcohols, e.g. cinnamic alcohol ((E)-3-phenylprop-2-en-1-ol); cis-3-hexenol ((Z)-hex-3-en-1-ol); citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl)pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl)pentan-2-ol); terpineol (2-(4-methylcyclohex-3-en-1-yl)propan-2-ol); or Timberol™ (1-(2,2,6-trimethylcyclohexyl)hexan-3-ol); 2,4,7-trimethylocta-2,6-dien-1-ol, and/or [1-methyl-2 (5-methylhex-4-en-2-yl)cyclopropyl]-methanol;
aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super® (1-(2,3,8,8-tetramethyl-1,2,3,4,5,6,7,8-octahydronaphthalen-2-yl)ethanone); Isoraldeine® ((E)-3-methyl-4-(2,6,6-trimethylcyclohex-2-en-1-yl)but-3-en-2-one); Hedione® (methyl 3-oxo-2-pentylcyclopentaneacetate); 3-(4-isobutyl-2-methylphenyl)propanal; maltol; methyl cedryl ketone; methylionone; verbenone; and/or vanillin;
ether, acetals and ketals, e.g. Ambrox® (3a,6,6,9a-tetramethyl-2,4,5,5a,7,8,9,9b-octahydro-1H-benzo[e][1]benzofuran); Amberketal® (3,8,8,11a-tetramethyldodecahydro-5H-3,5a-epoxynaphtho[2,1-c]oxepine), geranyl methyl ether ((2E)-1-methoxy-3,7-dimethylocta-2,6-diene); and/or Spirambrene® (2′,2′,3,7,7-pentamethylspiro[bicyclo[4.1.0]heptane-2,5′-[1,3]dioxane]);
esters and lactones, e.g. benzyl acetate; cedryl acetate ((1S,6R,8aR)-1,4,4,6-tetramethyloctahydro-1H-5,8a-methanoazulen-6-yl acetate); delta-decalactone (6-pentyltetrahydro-2H-pyran-2-one); Helvetolide® (2-(1-(3,3-dimethylcyclohexyl)ethoxy)-2-methylpropyl propionate); delta-undecalactone (5-heptyloxolan-2-one); and/or vetiveryl acetate ((4,8-dimethyl-2-propan-2-ylidene-3,3a,4,5,6,8a-hexahydro-1H-azulen-6-yl)acetate);
macrocycles, e.g. Ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
heterocycles, e.g. isobutylquinoline (2-isobutylquinoline).

Thus there is provided in a further aspect of the invention a fragrance composition comprising a compound of formula (I) (which encompasses compounds of formula (Ia), in particular a compound of formula (Ia) wherein R is methyl)).

In one particular embodiment the compound of formula (I) (which encompasses compounds of formula (Ia), in particular a compound of formula (Ia) wherein R is methyl) may be admixed with a compound of formula (IV)

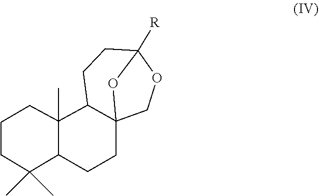

wherein R is selected from hydrogen, and C₁-C₄ alkyl (such as methyl, ethyl, n-propyl, and iso-propyl).

In one particular embodiment the compound of formula (IV) having the configuration of formula (IVa)

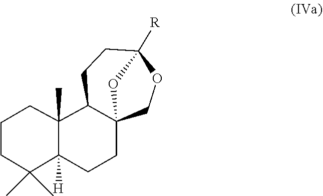

wherein R is selected from hydrogen, C₁-C₄ alkyl (such as methyl, ethyl, n-propyl, and iso-propyl).

In one particular embodiment the compound of formula (Ia) wherein R is methyl may be admixed with a compound of formula (IVa) wherein R is methyl (also known as (+)-Amberketal).

In one particular embodiment the compound of formula (Ia) wherein R is methyl may be admixed with (+)-Amberketal (which optionally might be obtained according to a method described in international patent application PCT/EP2021/059618 (WO2021/209482).

The compounds of formula (I) (which encompasses compounds of formula (Ia)) may be employed in a consumer product base simply by directly mixing the compound, or a fragrance composition comprising a compound of formula (I) (which encompasses compounds of formula (Ia)), or a mixture thereof, with the consumer product base, or it may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, and then mixed with the consumer product base.

Thus, the invention additionally provides a method of manufacturing a fragranced article, comprising the incorporation a compound of formula (I) (which encompasses compounds of formula (Ia)), or a mixture thereof as a fragrance ingredient, either by directly admixing to the consumer product base or by admixing a fragrance composition comprising a compound of formula (I) (which encompasses compounds of formula (Ia)), or a mixture thereof, which may then be mixed with a consumer product base, using conventional techniques and methods. Through the addition of an olfactory acceptable amount of a compound of formula (I) (which encompasses compounds of formula (Ia)), or a mixture thereof the odor notes of a consumer product base will be improved, enhanced, or modified.

Thus, the invention furthermore provides a method for improving, enhancing or modifying a consumer product base by means of the addition thereto of an olfactorily acceptable amount of a compound of formula (I) (which encompasses compounds of formula (Ia)), or a mixture thereof.

There is provided in a further aspect of the present invention a fragranced article comprising:
a) a compound of formula (I) (which encompasses a compound of formula (Ia))

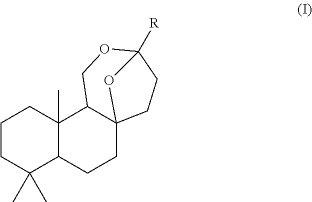

wherein R is selected from hydrogen, and C₁-C₄ alkyl (such as methyl, ethyl, n-propyl, and iso-propyl),
and
b) a consumer product base.

In one particular embodiment the compound of formula (I) (which encompasses a compound of formula (Ia)) is a compound wherein R is methyl.

As used herein, ‘consumer product base’ means a composition for use as a consumer product to fulfill specific actions, such as cleaning, softening, and caring or the like. Examples of such products include fine perfumery, e.g. perfume and eau de toilette; fabric care, household products and personal care products such as cosmetics, laundry care detergents, rinse conditioner, personal cleansing composition, detergent for dishwasher, surface cleaner; laundry products, e.g. softener, bleach, detergent; body-care products, e.g. shampoo, shower gel; air care products (includes products that contain preferably volatile and usually pleasant-smelling compounds which advantageously can even in very small amounts mask unpleasant odors). Air fresheners for living areas contain, in particular, natural and synthetic essential oils such as pine needle oils, citrus oil, eucalyptus oil, lavender oil, and the like, in amounts for example of up to 50% by weight. As aerosols they tend to contain smaller amounts of such essential oils, by way of example less than 5% or less than 2% by weight, but additionally include compounds such as acetaldehyde (in particular, <0.5% by weight), isopropyl alcohol (in particular, <5% by weight), mineral oil (in particular, <5% by weight), and propellants.

Cosmetic products include:
(a) cosmetic skincare products, especially bath products, skin washing and cleansing products, skincare products, eye makeup, lip care products, nail care products, intimate care products, foot care products;
(b) cosmetic products with specific effects, especially sunscreens, tanning products, de-pigmenting products, deodorants, antiperspirants, hair removers, and shaving products;
(c) cosmetic dental-care products, especially dental and oral care products, tooth care products, cleaners for dental prostheses, adhesives for dental prostheses; and
(d) cosmetic hair care products, especially hair shampoos, hair care products, hair setting products, hair-shaping products, and hair coloring products.

This list of products is given by way of illustration, and is not to be regarded as being in any way limiting.

In one particular embodiment the consumer product base is selected form fine perfumery, and personal care products, including deodorants, hair care products, soaps, and the like.

In a further particular embodiment the consumer product base is selected from fabric care products, including fabric softener, and home care products, including air fresheners, dish washers and the like.

The invention is now further described with reference to the following non-limiting examples. These examples are for the purpose of illustration only and it is understood that variations and modifications can be made by one skilled in the art.

Example 1: (5Z,8E and 5E,8E)-5-(2-hydroxyethylidene)-9,13-dimethyltetradeca-8,12-dien-2-one from a Mixture of (E)-2-(4,8-dimethylnona-3,7-dien-1-yl)-2-vinyloxirane/(E)-2-(6,10-dimethylundeca-1,5,9-trien-2-yl)oxirane Enriched Mixture in (E)-2-(6,10-dimethylundeca-1,5,9-trien-2-yl)oxirane

Step a) In a 25 ml two-neck flask equipped with a magnetic stirrer, a reflux condenser and a thermometer, a mixture of a 20:70 mixture of (E)-2-(4,8-dimethylnona-3,7-dien-1-yl)-2-vinyloxirane/(E)-2-(6,10-dimethylundeca-1,5,9-trien-2-yl)oxirane (1 g, 4.54 mmol), methyl acetoacetate (1.17 g, 9.98 mmol, 2.2 eq.), palladium (II) bis (acetylacetonate) (1.9 mg, 0.0091 mmol, 0.002 eq.), triphenylphosphine (9.6 mg, 0.036 mmol, 0.008 eq.), and potassium carbonate (1.28 g, 9.08 mmol, 2.0 eq.) in tetrahydrofuran (9 ml) was stirred at 40° C. for 24 h. The reaction mixture was cooled to room temperature, treated with water and extracted three time with EtOAc. The organic phases were combined, washed with aq. saturated NaCl solution, dried over Na₂SO₄, leading after solvent evaporation under reduced pressure to a crude mixture (1.9 g as a yellow oil).

Step b) In a 10 ml two-neck flask equipped with a magnetic stirrer, a reflux condenser and a thermometer, a solution of the crude product obtained in the previous step (1.89 g) in MeOH (5.3 ml) was treated with 2M aq. KOH (5.6 ml, 11.3 mmol) and heated at 40° C. for 6 h. The reaction mixture was cooled to room temperature, diluted with AcOEt, quenched with 2M aq. HCl and the aq. phase was extracted with AcOEt. The organic phases were combined, dried over MgSO₄, leading after solvent evaporation under reduced pressure wto a crude mixture (1.24 g as a brown oil; 1H-NMR: 20% (5Z,9E)-6-(hydroxymethyl)-10,14-dimethylpentadeca-5,9,13-trien-2-one and 80% of 5-(2-hydroxyethylidene)-9,13-dimethyltetradeca-8,12-dien-2-one as a 80:20 (5Z,8E)/(5E,8E)-mixture of isomers) that was purified by flash chromatography (heptane/AcOEt 1:0 to 2:8) on SiO₂ yielding first to a mixture of (5Z,9E)-6-(hydroxymethyl)-10,14-dimethylpentadeca-5,9,13-trien-2-one (56%) and isomers (5Z,8E)-/(5E,8E)-5-(2-hydroxyethylidene)-9,13-dimethyltetradeca-8,12-dien-2-one (44%, 2:1) (0.19 g, 15% yield) and to a second fraction) of 5-(2-hydroxyethylidene)-9,13-dimethyltetradeca-8,12-dien-2-one (0.48 g, 38% yield,) as 2:1 (5Z,8E)-/(5E,8E)-mixture.

(5Z,8E)-5-(2-hydroxyethylidene)-9,13-dimethyltetradeca-8,12-dien-2-one:

¹H NMR (400 MHz, CDCl₃) δ ppm 5.45 (t, J=7.2, 1H), 5.10-5.02 (m, 2H), 4.11 (d, J=7.1, 2H), 2.53 (t, J=7.4, 2H), 2.32 (t, J=7.4, 2H), 2.11 (s, 3H, MeCO), 2.10-1.90 (m, 8H), 1.65 (br. s, 3H), 1.57 (s, 6H).

¹³C NMR (100 MHz, CDCl₃) δ ppm 208.47 (s, 1C), 141.50 (s, 1C), 135.45 (s, 1C), 131.19 (s, 1C), 124.81 (d, 1C), 124.15 (d, 1C), 123.42 (d, 1C), 58.50 (t, 1C), 41.89 (t, 1C), 39.56 (t, 1C), 36.14 (t, 1C), 29.96 (q, 1C), 26.57 (t, 1C), 26.34 (t, 1C), 25.56 (q, 1C), 23.96 (t, 1C), 17.55 (q, 1C) 15.92 (q, 1C).

(5E,8E)-5-(2-hydroxyethylidene)-9,13-dimethyltetradeca-8,12-dien-2-one:

¹H NMR (400 MHz, CDCl₃) δ ppm 5.35 (br. t, J=6.9, 1H), 5.10-5.02 (m, 2H), 4.10 (d, J=6.9, 2H), 2.57-2.52 (m, 2H), 2.28 (t, J=7.1, 2H), 2.13 (s, 3H, MeCO), 2.10-1.90 (m, 8H), 1.65 (br. s, 3H), 1.57 (s, 6H).

¹³C NMR (100 MHz, CDCl₃) δ ppm 208.23 (s, 1C), 141.48 (s, 1C), 135.92 (s, 1C), 131.28 (s, 1C), 124.27 (d, 1C), 124.08 (d, 1C), 123.25 (d, 1C), 58.77 (t, 1C), 41.93 (t, 1C), 39.56 (t, 1C), 30.64 (t, 1C), 30.16 (t, 1C), 29.80 (q, 1C), 26.81 (t, 1C), 26.52 (t, 1C), 25.56 (q, 1C), 17.55 (q, 1C), 15.89 (q, 1C).

Mixture of (5Z,8E)- and (5E,8E)-5-(2-hydroxyethylidene)-9,13-dimethyltetradeca-8,12-dien-2-one:

GC-MS (EI): 260 (1, [M]^{+*}-H₂O), 217 (2), 209 (1), 202 (2), 191 (4), 175 (4), 159 (4), 149 (5), 133 (32), 121 (12), 105 (16), 93 (21), 91 (19), 81 (32), 79 (16), 69 (100), 67 (16), 55 (15), 43 (65), 41 (59).

### Example 2: SHC Biocatalyst Production

### SHC Plasmid Preparation

The gene encoding a wild-type or variant squalene hopene cyclase (SHC) enzyme was inserted into plas-mid pET-28a (+), where it is under the control of an IPTG inducible T7-promotor for protein production in Escherichia coli. The plasmid was transformed into E. coli strain BL21 (DE3) using a standard heat-shock transformation protocol.

### Biocatalyst Production in Fermenters

Fermentations were prepared and run in 750 ml InforsHT reactors. To the fermentation vessel was added 168 ml deionized water. The reaction vessel was equipped with all required probes (pO₂, pH, sampling, antifoam), C+N feed and sodium hydroxide bottles, and autoclaved. After autoclaving was added to the reactor:
20 ml 10× phosphate/citric acid buffer
14 ml 50% glucose
0.53 ml MgSO₄ solution
2 ml (NH₄)₂SO₄ solution
0.020 ml trace elements solution
0.400 ml thiamine solution
0.200 ml kanamycin stock

The running parameters were set as follows: pH=6.95, pO₂=40%, T=30° C., Stirring at 300 rpm. Cascade: rpm set point at 300, min 300, max 1000, flow (l/min) set point 0.1, min 0, max 0.6. Antifoam control: 1:9.

The fermenter was inoculated from a seed culture to an OD₆₅₀ₙₘ of 0.4-0.5. This seed culture was grown in LB medium (+Kanamycin) at 37° C., 220 rpm for 8 h. The fermentation was run first in batch mode for 11.5 h, where after was started the C+N feed with a feed solution (sterilized glucose solution (143 ml H₂O+35 g glucose) to which had been added after sterilization: 17.5 ml (NH₄)₂SO₄ solution, 1.8 ml MgSO₄ solution, 0.018 ml trace elements solution, 0.360 ml Thiamine solution, 0.180 ml kanamycin stock). The feed was run at a constant flow rate of approx. 4.2 ml/h. Glucose and NH₄⁺ measurements were done externally to evaluate availability of the C- and N-sources in the culture. Usually glucose levels stay very low.

Cultures were grown for a total of approximately 25 h, typically reaching an OD₆₅₀ₙₘ of 40-45. SHC production was then started by adding IPTG to a concentration of approx. 1 mM in the fermenter (as IPTG pulse or over a period of 3-4 hours using an infusion syringe), setting the temperature to 30° C. and pO₂ to 20%. Induction of SHC production lasted for 16 h at 40° C. At the end of induction the cells were collected by centrifugation, washed with 0.1 M citric acid/sodium citrate buffer pH 5.4 and stored as pellets at 4° C. or −20° C. until further use.

### Stock Solutions

Citric acid/phosphate stock: 133 g/l KH2PO4, 40 g/l (NH4)2HPO4, 17 g/l citric acid. H₂O with pH adjusted to 6.3) was added 307 ml H₂O, the pH adjusted to 6.8 with 32% NaOH as required.
Trace elements solution: 50 g/l Na₂EDTA·2H₂O, 20 g/l FeSO₄·7H₂O, 3 g/l H₃BO₃, 0.9 g/l MnSO₄·2H₂O, 1.1 g/l CoCl₂, 80 g/L CuCl₂, 240 g/l NiSO₄·7H₂O, 100 g/l Kl, 1.4 g/l (NH₄)₆Mo₇O₂₄·4H₂O, 1 g/l ZnSO₄·7H₂O, in deionized water.
Thiamin solution: 2.25 g/l Thiamin·HCl in deionized water.
MgSO₄ solution: 50% (w/v) MgSO₄·7H₂O in deionized water.
(NH₄)₂SO₄ solution: 50% (w/v) (NH₄)₂SO₄ in deionized water.

### Example 3—GC-analytics

Samples were extracted with an appropriate volume of tert-butylmethyl ether (MBTE/tBME) for quantification of their content in substrate and reaction products. The solvent fraction was separated from the water phase by centrifugation prior to analysis with gas chromatography. 1 μl of the solvent phase was injected (split ratio 10) onto a 30 m×0.32 mm×0.25 μm Agilent DB-5 column. The column was developed at constant flow (4 ml/min H₂) with the following temperature gradient: 100° C., 15° C./min to 200° C., 120° C./min to 240° C., 3 min at 240° C. Inlet temperature: 250° C., detector temperature: 250° C. This resulted in separation of substrate and product peaks.

Conversion was calculated from the areas of the peaks corresponding to substrate and reaction products with the following formula:

Conversion(%)=100×(Area_{Product Peaks}/(Area_{Product Peaks}+Area_{Substrate Peak(s)})

### Example 4—SHC Cyclization Reactions

Reactions were run using as biocatalyst E. coli cells that had produced the Alicyclobacillus acidocaldarius SHC enzyme variant SHC #65 (AacSHC #65), or the squalene hopene cyclase enzyme from Bacillus megaterium (BmeSHC).

Reactions (1 ml volume) contained 1 g/l substrate and E. coli cells that had produced the SHC enzyme of interest to and OD₆₅₀ₙₘ of 20, and SDS as a detergent. Reactions were run in citric acid sodium phosphate buffer pH 5.6 at 45° C. and under constant agitation (800 rpm, Heidolph Synthesis 1, Liquid 16). The reaction with AacSHC #65 contained 0.14% SDS, the reaction with BmeSHC contained 0.005% SDS. After 22 h of incubation, the reactions were extracted with 1 ml MTBE and analysed by GC-FID for their substrate and product content.

GC-FID profiles indicated the formation of two reaction products with the same major (P1) and same minor (P2) product in both reactions. Conversion was 15% in the reaction run with AacSHC #65 biocatalyst, and 70% in the reaction run with BmeSHC biocatalyst. The total reaction product (P1+P2) contained 87% P1 in the reaction run with SHC #65 and 98% P1 in the reaction run with BmeSHC.

### Preparative Scale Reaction

A 40 ml preparative scale reaction was done with 5.0 g/l 5-(2-hydroxyethylidene)-9,13-dimethyltetradeca-8,12-dien-2-one (Example 1) and 125 g/l BmeSHC biocatalyst. The reaction was run in 0.1 M succinic acid/NaOH buffer pH 5.6 at 45° C. in presence of 0.019% SDS, and under constant agitation (350 rpm, Radleys Monoblock Flask). Full conversion resulted in 20 h. The whole reaction was extracted 5 times each with 20 mL MTBE (tert-butylmethyl ether) with shaking for 15 min at 250 rpm on a lab-shaker, followed by centrifugation (15 min at 3′500 g). The MTBE fractions were pooled and dried over MgSO₄, filtered and the solvent evaporated. 0.3 g crude brown liquid product were obtained. The crude product was dissolved in 10 ml heptane, the solvent evaporated under nitrogen flow to a final volume of 1-2 ml volume and loaded onto a 5 g silica gel column (Biotage, Sfär Silica HC Duo 20 μm). Purification on the Biotage apparatus (heptane/MTBE) yielded 99 mg 30 of a white/yellowish solid crystalline material with product P1 of >95% purity. This material served for structure elucidation of P1.

P1=(5aS,7aS,11aS,11bS)-3,8,8,11a-tetramethyldodecahydro-3H-3,5a-epoxynaphtho[1,2-c]oxepine (compound of formula (Ia) wherein R is methyl)

¹H NMR (600 MHz, C₆D₆) δ ppm 3.88 (dd, J=11.3, 4.9, 1H), 3.63 (t, J=11.3, 1H), 2.14 (ddd, J=12.4, 9.4, 4.1, 1H), 1.97-1.88 (m, 2H), 1.82-1.72 (m, 3H), 1.60 (s, 3H), 1.46 (dm, J=13.9, 1H), 1.43-1.33 (m, 2H), 1.27-1.16 (m, 3H), 1.07-0.94 (m, 2H), 0.78 (dd, J=12.3, 2.0, 1H), 0.75 (s, 3H), 0.73 (dd, J=13.8, 3.8, 1H), 0.70 (s, 3H), 0.66 (s, 3H).

¹³C NMR (150 MHz, C₆D₆) δ ppm 104.44 (s, 1C), 83.59 (s, 1C), 60.80 (t, 1C), 55.70 (d, 1C), 54.01 (d, 1C), 41.82 (t, 1C), 39.20 (t, 1C), 38.79 (t, 1C), 36.30 (s, 1C), 36.09 (t, 1C), 33.06 (t, 1C), 33.55 (q, 1C), 32.99 (s, 1C), 24.72 (q, 1C), 21.78 (q, 1C), 19.86 (t, 1C), 18.46 (t, 1C), 15.61 (q, 1C).

GC-MS (EI): 278 (1), 263 (1), 248 (2), 233 (2), 218 (9), 203 (6), 190 (48), 175 (12), 164 (6), 149 (12), 140 (16), 133 (16), 123 (23), 109 (23), 107 (23), 105 (20), 99 (58), 95 (36), 93 (24), 91 (22), 81 (33), 79 (30), 69 (28), 67 (26), 55 (39), 43 (100), 41 (42), 29 (11).

Odor description (P1): woody, ambery, faint waxy fatty.

In addition, 3.2 mg of a yellow liquid with product P2 of only approx. 60% purity was isolated. The 3.2 mg crude P2 were dissolved in 250 μl heptane:isopropanol 95:5 and purified by semi-preparative HPLC. P2 purity in the two resulting fractions was of 93-94%. The solvent was evaporated under nitrogen flow resulting in 1.3 mg colourless liquid. This material served for structure elucidation of P2.

P2=4-((6aS, 10aS, E)-7,7,10a-trimethyl-5,6,6a,7,8,9,10,10a-octahydro-2H-benzo[b]oxocin-4-yl)butan-2-one (compound of formula (IIIa) wherein R=methyl)

¹H NMR (600 MHz, DMSO-d6) δ ppm 5.12 (s, 1H), 4.20 (br. d, J=18.4, 1H), 3.90 (br. d, J=18.4, 1H), 3.22 (br. t, J=12.0, 1H), 2.53-2.41 (m, 2H), 2.10 (br. t, J=7.5, 2H), 2.06 (s, 3H), 1.74 (td, J=12.8, 4.1, 1H), 1.66-1.35 (m, 6H), 1.28 (br. d, J=13.0, 1H), 1.15 (s, 3H), 1.18-1.11 (m, 2H), 0.87 (s, 3H), 0.81 (s, 3H).

¹³C NMR (150 MHz, DMSO-d6) δ ppm 208.00 (s, 1C), 134.90 (s, 1C), 124.56 (d, 1C), 78.97 (s, 1C), 60.90 (t, 1C), 44.96 (d, 1C), 41.69 (t, 1C), 41.57 (t, 1C), 34.97 (t, 1C), 34.21 (s, 1C), 33.78 (t, 1C), 33.01 (q, 1C), 29.67 (q, 1C), 27.51 (t, 1C), 24.98 (t, 1C), 22.77 (q, 1C), 21.79 (q, 1C), 19.65 (t, 1C).

GC-MS (EI): 278 (1), 260 (2), 245 (1), 220 (2), 202 (1), 187 (5), 175 (2), 137 (7), 136 (8), 124 (11), 123 (11), 109 (44), 107 (9), 95 (16), 93 (13), 91 (9), 81 (24), 79 (18), 69 (18), 67 (14), 58 (3), 55 (17), 43 (100), 41 (24), 29 (4).

Overall 100.3 mg reaction product (P1+P2) were recovered from 200 mg starting material engaged in the SHC cyclization reaction, which corresponds to an isolated yield of 50.1%.

### Example 5: Fragrance Composition for Fine Fragrance

parts by weight

| | parts by |
|---|---|
| | weight |
| Compound/Ingredient | 1/1000 |
| Ambrox ® | 3 |
| Bornyl Acetate (1,7,7-trimethylbicyclo<2.2.1>heptan- | 4 |
| 2-yl acetate) | |
| Cashmeran ® (6,7-dihydro-1,1,2,3,3-pentamethyl- | 10 |
| 4(5h)-indanone) | |
| Cedryl methyl ether | 75 |
| Dipropylene glycol (DPG) | 260 |
| Galaxolide ™ (1,3,4,6,7,8-hexahydro-4,6,6,7,8,8- | 150 |
| hexamethylindeno(5,6-c)pyran) | |
| Guaiacwood oil | 3 |
| Hedione ® | 120 |
| Iso E Super ® | 100 |
| Javanol ® ((1-methyl-2-(1,2,2- | 5 |
| trimethylbicyclo(3.1.0)-hex-3- | |
| ylmethyl)cyclopropyl)methanol)) | |
| Linalool (3,7-dimethyl-1,6-octadien-3-ol) | 40 |
| Muscenone ™ (3-methylcyclopentadecenone) | 5 |
| Patchouli oil | 3 |
| POLYSANTOL ((+/−).IT.TRANS.IT.-3,3- | 20 |
| DIMETHYL-5-(2,2,3-TRIMETHYL-CYCLOPENT-3-EN- | |
| 1-YL)-PENT-4-EN-2-OL) | |
| Safraleine ™ (2,3,3-trimethylindan-1-one) | 0.2 |
| Sandela ™ (4-(5,5,6-trimethylbicyclo(2.2.1)hept- | 100 |
| 2-yl)-cyclohexan-1-ol) | |
| Triethyl citrate (TEC) | 31.8 |
| Vetyvenal ® (CAS 57082-24-3) | 70 |
| Total: | 1000 |

The replacement of 30 parts of TEC of the accord above with 3,8,8,11a-tetramethyldodecahydro-3H-3,5a-epoxynaphtho[1,2-c]oxepine (a compound of formula (I) wherein R is methyl) @10% IPM (isopropyl myristate) brings depth to the woody ambery facet in particular and the overall accord in general. The addition of this molecule is key to the longaslintness and substantivity of said fragrance on skin.

SEQUENCE LISTING

SEQ ID NO: 1 (amino acid sequence of wild-type Bacillus megaterium SHC)
MIILLKEVQLEIQRRIAYLRPTQKNDGSFRYCFETGVMPDAFLIMLLRTFDLDKEVLIKQLTERIVSLQNEDGLW
TLFDDEEHNLSATIQAYTALLYSGYYQKNDRILRKAERYIIDSGGISRAHFLTRWMLSVNGLYEWPKLFYLPLSL
LLVPTYVPLNFYELSTYARIHFVPMMVAGNKKFSLTSRHTPSLSHLDVREQKQESEETTQESRASIFLVDHLKQL
ASLPSYIHKLGYQAAERYMLERIEKDGTLYSYATSTFFMIYGLLALGYKKDSFVIQKAIDGICSLLSTCSGHVHV
ENSTSTVWDTALLSYALQEAGVPQQDPMIKGTTRYLKKRQHTKLGDWQFHNPNTAPGGWGFSDINTNNPDLDDTS
AAIRALSRRAQTDTDYLESWQRGINWLLSMQNKDGGFAAFEKNTDSILFTYLPLENAKDAATDPATADLTGRVLE
CLGNFAGMNKSHPSIKAAVKWLFDHQLDNGSWYGRWGVCYIYGTWAAITGLRAVGVSASDPRIIKAINWLKSIQQ
EDGGFGESCYSASLKKYVPLSFSTPSQTAWALDALMTICPLKDQSVEKGIKFLLNPNLTEQQTHYPTGIGLPGQF
YIQYHSYNDIFPLLALAHYAKKHSS
SEQ ID NO: 2 (amino acid sequence of Alicyclobacillus acidocaldarius SHC enzyme
variant #65)
MAEQLVEAPAYARTLDRAVEYLLSCQKDEGYWWGPLLSNVTMEAEYVLLCHILDRVDRDRMEKIRRYLLHEQRED
GTWALYPGGPPDLDTTIEAYVALKYIGMSRDEEPMQKALRFIQSQGGIESSRVFTRRWLALVGEYPWEKVPMVPP
EIMFLGKRMPLNIYEFGSWARATVVALSIVMSRQPVFPLPERARVPELYETDVPPRRRGAKGGGGWIFDALDRVL
HGYQKLSVHPFRRAAEIRALDWLLERQAGDGSWGGIQPPWFYALIALKILDMTQHPAFIKGWEGLELYGVELDYG
GWMFQASISPVWDTGLAVLALRAAGLPADHDRLVKAGEWLLDRQITVPGDWAVKRPNLKPGGFAFQFDNVYYPDV
DDTAVVVWALNTLRLPDERRRRDAMTKGFRWIVGMQSSNGGWGAYDVDNTSDLPNHTPFCDFGEVTDPPSEDVTA
HVLECFGSFGYDDAWKVIRRAVEYLKREQKPDGSWFGRWGVNYLYGTGAVVSALKAVGIDTREPYIQKALDWVEQ
HQNPDGGWGEDCRSYEDPAYAGKGASTPSQTTWALMALIAGGRAESEAARRGVQYLVETQRPDGGWDEPYYTGTG
FPGDFYLGYTMYSHVFPTLALGRYKQAIERR

1. A method of method of making a compound of formula (I) 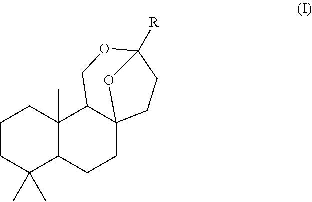
comprising the step of contacting a compound of formula (II) with a squalene-hopene cyclase (SHC) enzyme, 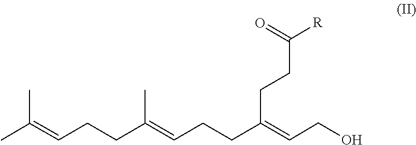
wherein R is selected from hydrogen, and C₁-C₄ alkyl.

2. The method according to claim 1 wherein R is methyl.

3. The method according to claim 1 wherein the compound of formula (II) is a mixture comprising E,Z-compound of formula (II), and E,E-compound of formula (II).

4. The method according to claim 1 comprising the step of contacting a mixture comprising a compound of formula (II) and at least one constitutional isomer thereof having the configuration of formula (IIa) 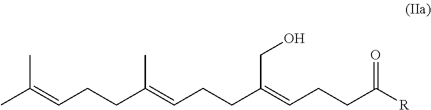
wherein R has the same meaning as for the compounds of formula (II).

5. The method according to claim 1 wherein the compound of formula (I) having the configuration of formula (Ia) 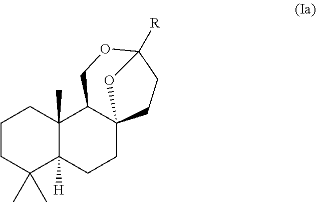
wherein R is selected from hydrogen, and C₁-C₄ alkyl.

6. A compound of formula (I) 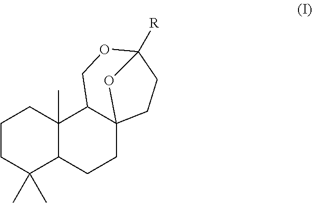
wherein R is selected from hydrogen, and C₁-C₄ alkyl.

7. The compound according to claim 6 having the configuration of formula (Ia) 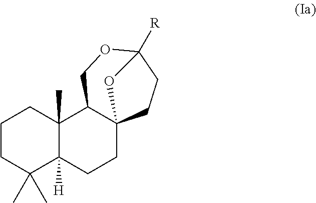
wherein R is selected from hydrogen, and C₁-C₄ alkyl.

8. The compound according to claim 6 wherein R is methyl.

9. A fragrance composition comprising a compound of formula (I) as defined in claim 6.

10. A fragranced article comprising:
a) a compound of formula (I) 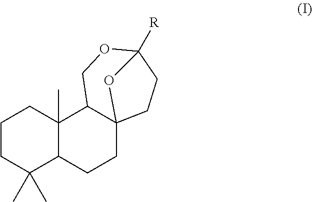
wherein R is selected from hydrogen, and C₁-C₄ alkyl. and
b) a consumer product base.

11. The fragranced article according to claim 10 wherein the compound of formula (I) having the relative configuration of formula (Ia) 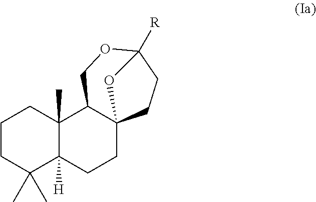
wherein R is selected from hydrogen, and C₁-C₄ alkyl.

12. The fragranced article according to claim 11 comprising a compound of formula (Ia) wherein R is methyl.

13. The fragranced article according to claim 10 further comprising a compound of formula (IV) 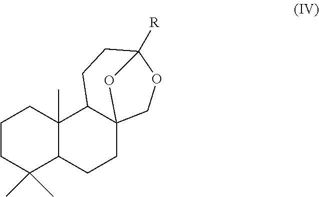
wherein R is selected from hydrogen, and C₁-C₄ alkyl.

14. The method according to claim 2 wherein the compound of formula (II) is a mixture comprising E,Z-compound of formula (II), and E,E-compound of formula (II).

15. The method according to claim 2 comprising the step of contacting a mixture comprising a compound of formula (II) and at least one constitutional isomer thereof having the configuration of formula (IIa) 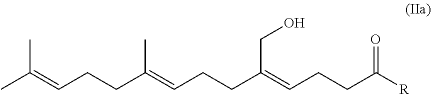
wherein R has the same meaning as for the compounds of formula (II).

16. The method according to claim 3 comprising the step of contacting a mixture comprising a compound of formula (II) and at least one constitutional isomer thereof having the configuration of formula (IIa) 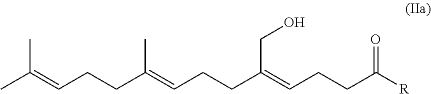
wherein R has the same meaning as for the compounds of formula (II).

17. The method according to claim 2 wherein the compound of formula (I) having the configuration of formula (Ia) 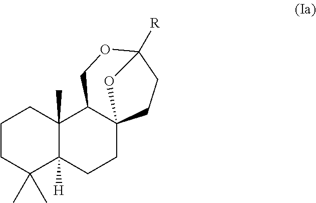
wherein R is selected from hydrogen, and C₁-C₄ alkyl.

18. The method according to claim 3 wherein the compound of formula (I) having the configuration of formula (Ia) 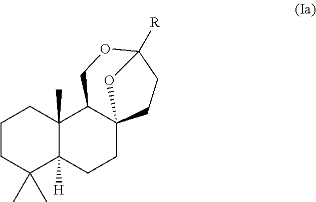
wherein R is selected from hydrogen, and C₁-C₄ alkyl.

19. The method according to claim 4 wherein the compound of formula (I) having the configuration of formula (Ia) 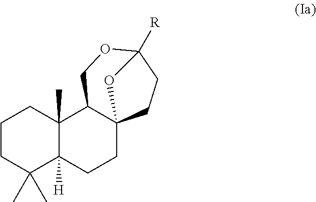
wherein R is selected from hydrogen, and C₁-C₄ alkyl.

20. The compound according to claim 7 wherein R is methyl.


SEQUENCE LISTING (2 sequences)

SEQ ID NO: 1 - protein, 625 aa, synthetic construct
  source 1..625: note = Bacillus megaterium wt SHC enzyme
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 2 - protein, 631 aa, synthetic construct
  source 1..631: note = amino acid sequence of Alicyclobacillus acidocaldarius SHC enzyme variant SHC#65
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTTWALMALIAGGRAESEAARRGVQYLVETQRPDGGWDEPYYTGTGFPGDFYLGYTMYSHVFPTLALGRYKQAIERR